(19) United States
(12) Patent Application Publication
(10) Pub. No.: US 2020/0305774 A1
(43) Pub. Date: Oct. 1, 2020

(54) APPARATUS FOR DIAGNOSIS OF DIABETIC FOOT

(71) Applicant: NEUBOURG SKIN CARE GMBH & CO. KG, Greven (DE)

(72) Inventor: Alois CREMERIUS, Dorsten (DE)

(21) Appl. No.: 16/464,139

(22) PCT Filed: Jan. 16, 2017
(86) PCT No.: PCT/EP2017/050788

§ 371 Date: May 24, 2019

(51) Int. Cl. A61B 5/145; A61B 5/00; A61B 5/053

(52) U.S. Cl. CPC A61B 5/14532; A61B 5/0051; A61B 2560/0214; A61B 5/0531; A61B 2562/166; A61B 5/6829

(57) ABSTRACT

The invention relates to a device for the diagnosis of a diabetic foot, comprising at least one first unit for generating vibrations. The device is to be compact and easy to handle. According to the invention, the first unit has an electric motor (5), wherein a weight (14) is secured eccentrically on the shaft thereof such that vibrations of a predetermined strength are generated.

The invention relates to an apparatus for diagnosing diabetic foot, comprising at least a first means for generating vibrations.

In diabetics, there is a high risk of developing diabetic foot, meaning poor circulation of the extremities with its negative consequences. It is therefore important to recognize diabetic foot early in at-risk patients and then treat them.

Apparatuses for diagnosing diabetic foot are generally known and include, for example, means for testing warm/cold sensitivity (also referred to as warm/cold perception or cold/warm discrimination), electrical skin resistance as a measure of skin moisture, or vibration thresholds.

Each such apparatuses monitors contains only a single of these functions.

An instrument for determining the electrical resistance of skin, for example, is known from DE 92 06 428 U1 in which an LCD (liquid crystal display) is used to display the measured value to the input of which a particular resistor is connected in parallel. The apparatus is relatively elaborate and bulky and too complicated for use by a patient.

EP 2 898 834 [US 2015/0182158] discloses an apparatus for detecting diabetic foot (neuropathy) using a threshold value that compares measured values with reference values. The apparatus is very bulky and complicated and can only be operated by qualified personnel.

Tuning forks are still known to determine the threshold value.

It is the object of the invention to provide an apparatus for diagnosing diabetic foot that is compact and easy to handle.

The object is achieved by the features of claim 1. The first means comprises an electric motor having a shaft to which a weight is eccentrically mounted such that the vibrations are generated in a predetermined intensity during operation of the electric motor. Here, the intensity of the vibrations is synonymous with the amplitude and is a measure of the palpability of the vibration on the skin. The electric motor with the eccentric weight makes it easy to detect a vibration threshold that a person (patient) feels or does not feel. The apparatus is very easy to handle by switching on the electric motor and then placing the apparatus in different places on a person's foot. That person recognizes and, as applicable, communicates to an examining person whether or not he feels the vibration. The apparatus can be made very compact, for example in the form of a pen, which further facilitates handling. The pen has a length of 10 cm and a diameter of 15 mm, for example; the weight is less than 50 g. The patient is able to perform the examination independently.

Moreover, the apparatus is inexpensive to produce, so that self-monitoring by patients makes sense for this reason as well.

The subclaims relate to advantageous embodiments of the invention.

In one embodiment, the vibrations have a frequency of 64 Hz. This represents the standard frequency for this investigation. It is ensured by a number of revolutions of the electric motor of 3840 rpm.

In another embodiment, a rear longitudinal end of the apparatus is made of plastic and a front end of metal. At objectively the same temperature, metal feels subjectively colder than plastic. By placing the different ends on the examined skin areas of the person, it is thus also possible to examine that person's temperature perception—warm or cold—and thus particularly their ability to discriminate temperatures.

In another embodiment, the apparatus has a second means for detecting skin resistance. This enables the apparatus to perform a third—here objective—exploratory examination for diabetic foot.

In another embodiment, predetermined value ranges of skin resistance are assigned to light sources that emit a predetermined color for each of a plurality of value ranges. The value ranges are associated with normal skin, an intermediate range, and excessively dry skin, for example; the corresponding colors are green, yellow, and red. As a result, the measurement results are easy to interpret even for a medical layperson.

In another embodiment, the apparatus includes a printed circuit board with electrical and/or electronic components for control and/or evaluation. As a result, all of the required electrical and electronic functions are provided in a compact form.

In another embodiment, the apparatus has a pushbutton for switching electrical functions. The pushbutton allows the apparatus to be switched on and switched to the vibration test or skin resistance measurement, or it can be switched off. Only a single pushbutton is required instead of multiple switches.

In another embodiment, contacts for detecting skin resistance are also designed for charging a rechargeable battery. This facilitates the charging of the battery by a charger without negatively impacting the handiness of the apparatus. What is more, manufacture of the apparatus is simplified.

In another embodiment, the contacts are integrated into the front end and a switch knob into the rear end. The contacts do not need to be insulated separately. Unintentional switching is avoided.

In another embodiment, the apparatus is configured to perform a self-test. This ensures reliable operation of the apparatus; incorrect measurements are prevented.

The invention will be explained in further detail with reference to the schematic drawing, in which:

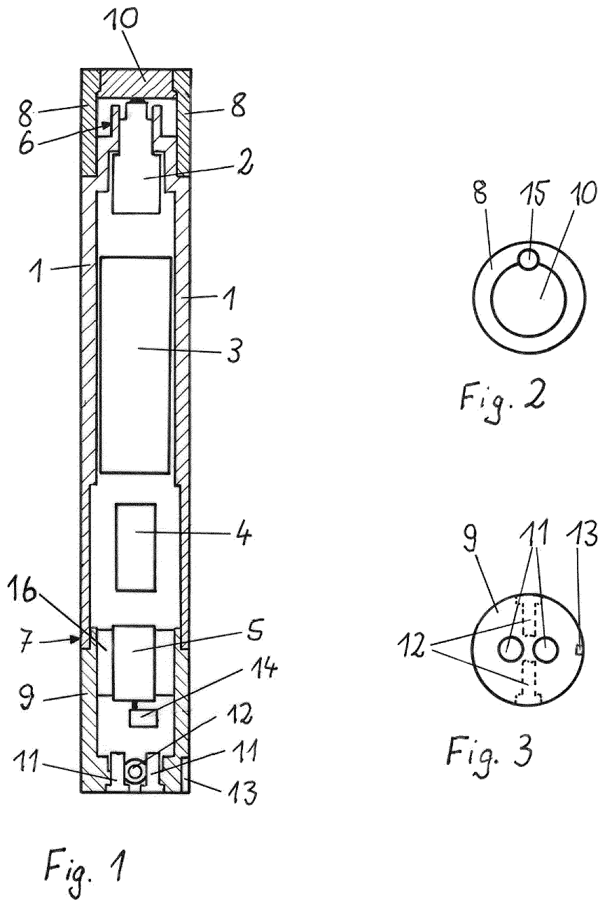

FIG. 1 is a longitudinal section through an apparatus according to the invention,

FIG. 2 is a plan view of detail of a rear end of the apparatus, and

FIG. 3 is a plan view of detail of a front end of the apparatus.

The embodiment relates to an apparatus with which three tests for diagnosing diabetic foot can be carried out: heat sensitivity, vibration threshold, and skin resistance.

As can be seen from FIGS. 1 to 3, the apparatus comprises a housing 1 of circular cross section. A pushbutton 2, a printed circuit board 3 with electrical and electronic components, a rechargeable battery 4, and an electric motor 5 are mounted in or on the housing 1.

The housing 1 is largely hermetically sealed by rear and front caps 8 and 9 at its rear and front ends, i.e. at a rear longitudinal end 6 of the apparatus shown at the top in FIG. 1 and at a front longitudinal end 7 situated opposite the rear end 6.

A rear cap 8 on the rear end 6 is made of metal such as V2A and fastened to the rear end 6 by a screw thread or interference fit. A switch knob 10 for actuating the pushbutton 2 is integrated into the rear cap 8 such that, when the unactuated switch knob 10 is in a rest position, a flush rear end face is formed on the apparatus. The switch knob 10 is guided loosely in the rear cap 8 and held by a spring force of the pushbutton 2 on a stepped stop. The switch knob 10 is made of the same metal as the rear cap 8.

At least one light source 15 is in the rear cap 8 [front cap 9] such that its light emission takes place at the rear [front] end face. Either three light sources 15 are set up, in which case each emits a different color, or preferably a single light source 15 is a three-color LED (light-emitting diode), a so-called RGB LED. The emitted color is green, yellow, or red. The light source is electrically connected to the printed circuit board 3.

In an alternative embodiment, the light source 15 is on the printed circuit board 3, and the light is conducted by an optical waveguide to the front end face.

A front cap 9 on the front end 7 is made of plastic and fixed to the housing 1 by an interference fit. Two measuring contact pins 11 are fastened in the front end face of the front cap 9. These are circular in cross section and enlarged in stepped fashion at one end. A first diameter of the measuring contact pins 11 is 2 mm, and a second diameter is 3.5 mm. The enlarged end of each of the measuring contact pins 11 terminates flush with the front end face of the apparatus. The measuring contact pins 11 extend parallel to a longitudinal axis of the apparatus; they have a lateral spacing of 7 mm and are electrically connected to the printed circuit board 3.

Furthermore, two charging contact pins 12, which serve as measuring contact pins 11 and are electrically connected to the printed circuit board 3, are fastened in the front cap 9. The charging contact pins 12 are extend radially outward from the longitudinal axis of the apparatus opposite one another, that is with an angular separation of 180°.

Starting from the front end face, a groove 13 sunk laterally into the front cap 9 extends parallel to the longitudinal axis of the apparatus. The groove 13 is from 3 mm to 10 mm long and is angularly offset from the charging contact pins 12 by 90°. The groove 13 corresponds to a complementary rib of a charger for charging the rechargeable battery 4 in order to ensure a correct polarity for the charging process.

In an alternative embodiment, the measuring contact pins 11 also serve as the charging contact pins 12, so that only a single pair of contact pins 11, 12 is provided.

The electric motor 5 is held in a mount 16 in the front cap 9 such that an forwardly extending end of its output shaft extends toward a rear face of the front cap 9. A weight 14 is mounted eccentrically at this end of the shaft. The mount 16 has on its outer side at least one axially throughgoing groove for the passage of electrical conductors from the contact pins 11, 12 to the printed circuit board 3. The electric motor is electrically connected to the printed circuit board 3.

Near the rear end 6, the pushbutton 2 is fastened in the housing 1 such that it can be actuated by the switch knob 10 and holds sam in the intended position. The pushbutton 2 is electrically connected to the printed circuit board 3 fixed according to FIG. 1 forward of the pushbutton 2 in the housing 1.

The rechargeable battery 4 is fixed in the housing 1 between the printed circuit board 3 and the electric motor 5 and electrically connected to the circuit board 2.

A clip for releasably securing the apparatus to a shirt or lab coat pocket, for example, is preferably fastened to the outside of the apparatus.

The first means for generating vibrations comprises at least the housing 1, the caps 8, 9, the switch knob 10, the pushbutton 2, the printed circuit board 3, the rechargeable battery 4, and the electric motor 5 with the weight 14.

The second means for detecting skin resistance comprises, in addition to the first means, the measuring contact pins 11 and the light source 15.

But it is also possible for the second means to be a separate device. It then comprises the housing 1, the caps 8, 9, the switch knob 10, the pushbutton 2, the printed circuit board 3, the rechargeable battery 4, the measuring contact pins 11, and the light source 15.

To diagnose diabetic foot, the apparatus is used as follows, the order being arbitrary:

In order to examine the temperature perception, the two end faces of the apparatus are variously placed at different locations of the foot of a person to be examined. The person is asked each time about the sensation, and the result is noted and evaluated.

In order determine the vibration threshold, the apparatus and hence the electric motor 5 is switched on by a single actuation of the switch knob 10. A red light indicates operation. The front end face is placed on different parts of the foot. The person is asked each time about the sensation, and the result is noted and evaluated.

To examine skin resistance, the apparatus is switched appropriately by double-clicking the switch knob 10. A blinking red light indicates operation. The front end face is placed on different parts of the foot. Correspondingly colored light indicates the condition of the skin: red for excessively dry skin, yellow for a transitional range, and green for normal skin.

It is possible to switch between the two operating modes by double-clicking.

The apparatus is switched off either automatically after a predetermined time or by prolonged pressing of the switch knob 10.

If the apparatus is not needed for a long time, it is inserted into the charger, thereby charging the rechargeable battery 4.

The apparatus performs a self-test automatically at regular intervals.

The apparatus can also be used by the person to be examined for regular self-monitoring.

List of reference symbols

| | |
|---|---|
| 1 | housing |
| 2 | pushbutton |
| 3 | printed circuit board |
| 4 | accumulator |
| 5 | electric motor |
| 6 | rear end |
| 7 | front end |
| 8 | rear cap |
| 9 | front cap |
| 10 | switch knob |
| 11 | measuring contact pin |
| 12 | charging contact pin |
| 13 | groove |
| 14 | weight |
| 15 | light source |
| 16 | mount |

1-10. (canceled)

11. An apparatus for diagnosing diabetic foot, the apparatus comprising:
a housing;
first means in the housing for generating vibrations and including an electric motor having a shaft carrying an eccentric weight such that rotation of the shaft vibrates the housing; and
second means in the housing for detecting skin resistance.

12. The apparatus defined in claim 11, wherein the vibrations have a frequency of 64 Hz.

13. The apparatus defined in claim 11, wherein a rear longitudinal end of the housing is made of plastic and a front end thereof is made of metal.

14. The apparatus defined in claim 11, wherein the second means includes a plurality of light sources that emit respective colors for each of a plurality of value ranges.

15. The apparatus defined in claim 11, further comprising:
a printed circuit board with electrical and/or electronic components for control and/or evaluation.

16. The apparatus defined in claim 15, further comprising:
a pushbutton for switching electrical functions.

17. The apparatus defined in claim 15, further comprising:
a rechargeable battery connected to the printed-circuit board, the second means including contact pins for detecting skin resistance and also serving for charging the rechargeable battery.

18. The apparatus defined in claim 17, wherein the contacts are integrated into a front end of the housing and a switch knob is carried on a rear end thereof.

19. The apparatus defined in claim 15, wherein the printed-circuit board is configured to carry out a self-test.
